(19) United States
(12) Patent Application Publication
(10) Pub. No.: US 2011/0218572 A1
(43) Pub. Date: Sep. 8, 2011

(54) EXPANDABLE LAMINA SPINAL FUSION IMPLANT

(76) Inventors: Beat Lechmann, Langendorf (CH); Laura Villiger, Langendorf (CH)

(21) Appl. No.: 13/033,145

(22) Filed: Feb. 23, 2011

Related U.S. Application Data
(60) Provisional application No. 61/310,492, filed on Mar. 4, 2010.

(51) Int. Cl. A61B 17/70; A61B 17/56; A61B 17/88

(52) U.S. Cl. 606/249; 606/86 A; 606/279

(57) ABSTRACT

An expandable intervertebral implant comprises a caudal fixator including a caudal fixator body and a socket extending longitudinally upward from the caudal fixator body, a cranial fixator including a cranial fixator body and a core extending longitudinally downward from the cranial fixator body, and a circlip configured to fix the longitudinal position of the caudal fixator relative to the cranial fixator. The core can include outwardly-extending cranial ratchet ridges and can be configured to fit into the socket. The circlip can include inwardly-extending circlip ratchet ridges and can be configured to fit inside the socket. The implant can be configured to be installed into an intervertebral space between vertebrae of the spinal motion segment by attaching the implant to laminae of the vertebrae. The implant can be configured to be expanded after installation into the spinal motion segment, such that the implant extends between spinous processes of the vertebrae.

### CROSS REFERENCE TO RELATED APPLICATIONS

This application claims the benefit of U.S. Provisional Patent Application Ser. No. 61/310,492 filed Mar. 4, 2010, the disclosure of which is hereby incorporated by reference as if set forth in its entirety herein.

### BACKGROUND

Degenerative disc disease or degeneration of a vertebral body often results in a loss of disc height, which in turn can cause facet and nerve impingement, among other diseases which might create pain or inflammatory reaction.

Conventional posterior lumbar fusion is typically performed using translaminar screws or pedicle screw fixation. The preparation of the pedicles to provide screw entry points is extensively invasive. For instance, the erector muscles are typically dissected from the spinal segments, thereby compromising the physiological integrity of the spinal region. The preparation of the pedicles can also cause the patient to experience significant residual postoperative pain.

Furthermore, while surgical fixation of the spine can be effective to relieve immediate pain and symptoms associated with the degenerative condition, the surgical fixation does not eliminate or stop the degenerative process. As a result, subsequent surgical procedures can become necessary to address continued degeneration. However, the fixation of pedicle screws to the pedicle for posterior lumbar fixation can cause the pedicles to become biomechanically compromised for a later revision treatment. As a result, subsequent, more extensive and invasive, procedures often include cement augmentation, application of bone morphogenetic proteins (BMPs), larger pedicle screws, and the like.

Other methods of performing lumbar fusion include the application of translaminar screws, which include the insertion of anterior vertebral interbody spacers in order to maintain segmental stiffness. While the translaminar screws may block the facet joint, this method still allows a slight opening of the motion segment if patient movement causes the spine to extend as described in Mueller M E: Manual of internal fixation: techniques recommended by the AO-ASIF Group, 3^{rd} issue 1991, page 660ff. As described in Oxland T R, Lund T. Biomechanics of stand-alone cages and cages in combination with posterior fixation: a literature review. Eur Spine J. 2000; 9 Suppl 1:S95-101, translaminar screw fixation may be combined with an intervertebral spacer, such as an ALIF Cage, in order to reduce or even avoid the collapse of the intervertebral space.

### SUMMARY

An expandable intervertebral implant for posterior lumbar intervertebral fusion of a spinal motion segment and a method of expanding an intervertebral implant for posterior lumbar intervertebral fusion of a spinal motion segment are disclosed.

An expandable intervertebral implant configured to be inserted into an intervertebral space defined between first and second vertebrae is disclosed. The implant may include a first fixator and a second fixator. The first fixator may include a first fixator base, and may be configured to be attached to a lamina of the first vertebra. The second fixator may include a second fixator base, and may be configured to be attached to a lamina of the second vertebra. The implant may also include a socket extending out from the second fixator base and a core extending out from the first fixator base and sized to be received in the socket. The core may include an engagement member configured to releasably fix a position of the first fixator relative to the second fixator.

The implant may also include a circlip configured to fix the longitudinal position of the second fixator relative to the first fixator. The circlip may include an engagement member and can be configured to fit inside the socket. The circlip engagement member can be configured to mate with the engagement member of the core. The implant may be configured to be installed into an intervertebral space between vertebrae of the spinal motion segment by attaching the implant to laminae of the vertebrae. The implant may be configured to be expanded after installation into the spinal motion segment, such that the implant extends between spinous processes of the vertebrae.

In another embodiment an expandable intervertebral implant system comprising an intervertebral implant and an insertion device is disclosed. The intervertebral implant may be configured to be inserted into an intervertebral space defined between adjacent vertebrae and attached to a spinous process of the adjacent vertebrae. The implant may include a first fixator, a second fixator, and a locking mechanism that selectively allows the first and second fixators to expand from a first height to a second height. The insertion device may be configured to be coupled to the implant. The insertion device may include an actuator that is configured to selectively engage the locking mechanism so as to selectively unlock the locking mechanism and allow the first and second fixators to expand from the first height to the second height.

A method of expanding an intervertebral implant for posterior lumbar intervertebral fusion of a spinal motion segment includes the steps of inserting the implant into an insertion device, inserting the implant into an intervertebral space between vertebrae of the spinal motion segment, attaching a second fixator of the implant to a lamina of a first vertebra of the vertebrae, widening a circlip such that inwardly-extending ratchet ridges of the circlip are disengaged from outwardly-extending ratchet ridges of a core of a first fixator of the implant, translating the first fixator relative to the second fixator, releasing the circlip to engage the ratchet ridges of the circlip into the ratchet ridges of the first fixator core, and attaching the first fixator to a lamina of a second vertebrae.

### BRIEF DESCRIPTION OF THE DRAWINGS

FIG. 1 is a perspective view of an intervertebral implant according to an exemplary embodiment, installed in an intervertebral space;

FIG. 2A is a partially-transparent top view of the intervertebral implant installed in an intervertebral space depicted in FIG. 1;

FIG. 2B is a partially-transparent side view of the intervertebral implant installed in an intervertebral space depicted in FIG. 1;

FIG. 2C is a backside view of the intervertebral implant installed in an intervertebral space depicted in FIG. 1;

FIG. 3A is a right perspective view of the intervertebral implant depicted in FIG. 1;

FIG. 3B is a left perspective view of the intervertebral implant depicted in FIG. 3A;

FIG. 3C is an exploded perspective view of the intervertebral implant depicted in FIG. 3A;

FIG. 4A is a top perspective view of a first fixator of the intervertebral implant depicted in FIG. 3A;

FIG. 4B is a partial side perspective cross-sectional view of the intervertebral implant depicted in FIG. 3B, taken along the lines 4B-4B;

FIG. 5A is a rear perspective view of portions of the intervertebral implant depicted in FIG. 3A, showing a range of poly-axial insertion directions of bone screws adapted to affix the intervertebral implant to the vertebral laminae;

FIG. 5B is a side elevation view of a bone screw depicted in FIG. 5A;

FIG. 5C is an enlarged perspective view of a screw insertion aperture of the intervertebral implant depicted in FIG. 5A;

FIG. 6 is a rear view of the treated area in a patient, shown without soft tissue, showing the median incision and stab incisions configured for insertion of the intervertebral implant depicted in FIG. 3A;

FIG. 7A is a side perspective view of the intervertebral implant installed in an intervertebral space depicted in FIG. 1, held by an insertion device according to an exemplary embodiment;

FIG. 7B is a close-up side perspective view of an expandable body of the insertion device depicted in FIG. 7A;

FIG. 7C is a close-up side perspective view of the intervertebral implant installed in an intervertebral space depicted in FIG. 1, being held by the expanding tip of the insertion device depicted in FIG. 7A;

FIG. 8A is an exploded view of the intervertebral implant depicted in FIG. 3A, and the insertion device depicted in FIG. 7A;

FIG. 8B is a right perspective cross-sectional view of the intervertebral implant held by the insertion device depicted in FIG. 8A;

FIG. 9A is a perspective view of the intervertebral implant installed in an intervertebral space depicted in FIG. 1, held by the insertion device depicted in FIG. 7A, shown with the tip of a bone drill positioned to drill a hole into the lamina of a vertebra, through an aperture in a drill aiming device;

FIG. 9B is a perspective view of the drill aiming device depicted in FIG. 9A;

FIG. 9C is a perspective view of the tip of the drill aiming device depicted in FIG. 9B, showing a range of poly-axial drilling directions;

FIG. 9D is a perspective view of the intervertebral implant installed in an intervertebral space depicted in FIG. 1, held by the insertion device depicted in FIG. 7A, shown with the tip of a screwdriver drilling a bone screw into a hold in the lamina of a vertebra, through an aperture in the intervertebral implant;

FIG. 10A is a perspective view of an intervertebral implant including a spring, according to another embodiment;

FIG. 10B is a perspective view of an intervertebral implant including an elastic dampening device, according to another embodiment;

FIG. 11A is a partial side cross-sectional view of a bone screw including a poly-axial fixation mechanism, the bone screw suitable for use in installing any of the intervertebral implant embodiments; and

FIG. 11B is a front cross-sectional view of the intervertebral implant depicted in FIG. 3B, including four bone screws depicted in FIG. 11A.

### DETAILED DESCRIPTION OF ILLUSTRATIVE EMBODIMENTS

Certain terminology is used in the following description for convenience only and is not limiting. The words “right”, “left”, “lower” and “upper” designate directions in the drawings to which reference is made. The words “inwardly” or “distally” and “outwardly” or “proximally” refer to directions toward and away from, respectively, the geometric center of the expandable implant, instruments and related parts thereof. The words, “anterior”, “posterior”, “superior,” “inferior” and related words and/or phrases designate preferred positions and orientations in the human body to which reference is made and are not meant to be limiting. The terminology includes the above-listed words, derivatives thereof and words of similar import.

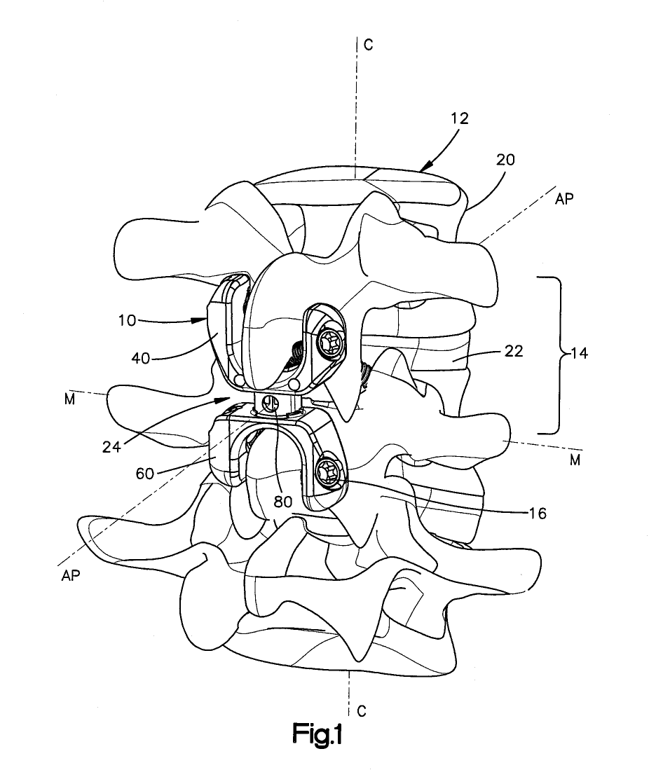

Referring to FIG. 1, an expandable intervertebral implant 10 for posterior lumbar intervertebral fusion is shown installed into a vertebral column 12 for stiffening or stabilizing a spinal motion segment 14. The vertebral column 12 includes a plurality of vertebrae 20, each adjacent pair of vertebrae 20 separated by an intervertebral disc 22 and defining an intervertebral space 24 therebetween. The implant 10 includes a first or cranial fixator 40 and a second or caudal fixator 60 that is moveable relative to the cranial fixator 40, and a circlip 80 that is configured to fix the longitudinal position of the caudal fixator 60 relative to the cranial fixator 40. The implant 10 is installed into the intervertebral space 24, and the implant 10 is attached to the vertebrae 20 by bone screws 16. The implant 10 can be configured to fuse with the vertebrae 20.

The vertebrae 20 can be disposed in any vertebral region as desired, and is illustrated in the lumbar region defining an anterior side AS and an opposing posterior side PS that are disposed on opposing sides of an central anterior-posterior axis AP-AP that extends along an anteroposterior direction. The vertebrae 20 further define opposing lateral sides LS that are disposed on opposing sides of a central medial axis M-M that extends along a mediolateral direction. The vertebrae 20 are illustrated as being spaced along a caudocranial axis C-C. The implant 10 extends generally along a longitudinal direction L, a lateral direction A, and a transverse direction T.

Various structure is therefore described as extending vertically along a longitudinal direction “L,” and horizontally along a lateral direction “A” and a transverse direction “T”. The intervertebral implant 10 is expandable in the longitudinal direction L. Unless otherwise specified herein, the terms “longitudinal,” “lateral,” and “transverse” are used to describe the orthogonal directional components of various components. The directional terms “inboard” and “inner,” “outboard” and “outer,” and derivatives thereof are used herein with respect to a given apparatus to refer to directions along the directional component toward and away from the geometric center of the apparatus.

It should be appreciated that while the lateral and transverse directions are illustrated as extending along a horizontal plane, and that the longitudinal direction is illustrated as extending along a vertical plane, the planes that encompass the various directions may differ during use. Accordingly, the directional terms “vertical” and “horizontal” are used to describe the intervertebral implant 10 and its components as illustrated merely for the purposes of clarity and illustration.

In the illustrated embodiment, the longitudinal direction L extends in the caudocranial direction, the lateral direction A extends in the mediolateral direction, and the transverse direction T extends in the anteroposterior direction. It should be appreciated, however, that the directions defined by the expandable intervertebral implant 10 could alternatively be oriented at various angles between 0° and 180° with respect to the various directions defined by the vertebrae 20. For instance, the lateral and transverse directions of the implant could be oriented at various angles between 0° and 180° with respect to the mediolateral and anteroposterior directions. As will become appreciated from the description below, the intervertebral implant 10 can be inserted into the intervertebral space 24 in an anterior direction, a posterior direction, or various alternative directions between 0° and 180° with respect to the anterior and posterior sides.

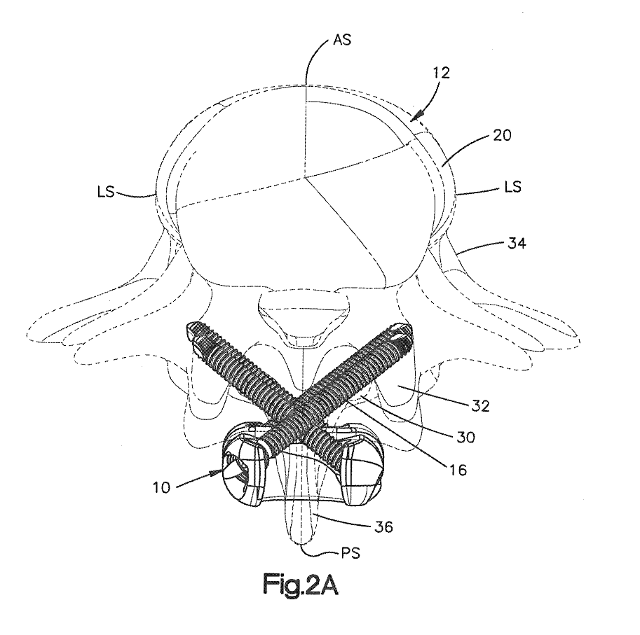

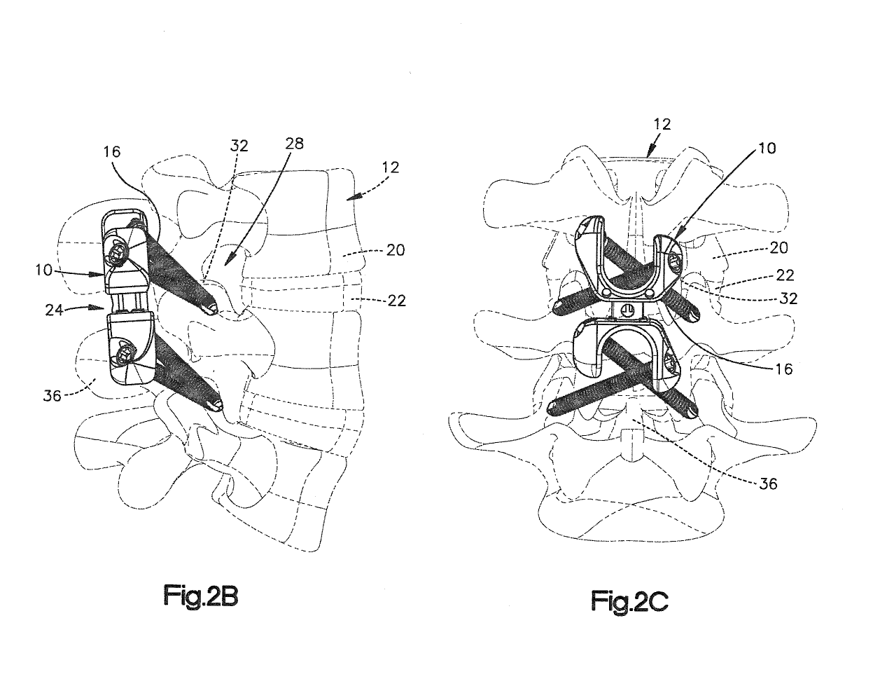

Referring now to FIGS. 2A-2C, the implant 10 can be attached to a bony structure of the vertebrae 20, for instance at the posterior end of the vertebrae 20, such as the spinous process 36, by inserting the bone screws 16 into the vertebrae 20, for instance into the laminae 30 of the vertebrae 20. As illustrated, the bone screws 16 can have sufficient length to penetrate the facet joint 32 between the laminae 30 of the two vertebrae 20 adjacent to the implant 10, or, alternatively, the bone screws 16 can be shorter, such that they do not penetrate the facet joint 32.

The length of the bone screws 16 can be chosen as desired to determine the degree of stability that the implant 10 provides to the spinal motion segment 14. If shorter bone screws 16 are used that do not penetrate the facet joint 32, the spinal motion segment 14 can have limited stability (i.e., some residual motion remains after the implant 10 is installed, in particular for the intervertebral space where an intact disc might be present) that results in posterolateral fusion. If longer bone screws 16 are used that penetrate the facet joint 32, the spinal motion segment 14 may be stiffened, such that there will be a high chance of circumferential fusion (i.e., including the intervertebral disc 22). With either type of fusion, the bone screws 16 avoid penetrating into the vertebral foramen 26 and the neural foramen 28.

Use of the pedicles 34 of the vertebrae 20 for attaching the implant 10 to the vertebrae 20 is avoided, thereby leaving the pedicles 34 available for future treatment in the event of further spine degeneration. As described above, when the pedicles 34 are used to attach a first implant, the pedicles 34 can be bio-mechanically compromised for a later revision treatment, so later revisions may require, for example, cement augmentation, application of bone morphogenetic proteins (BMPs), or use of larger screws. Use of the laminae 30 of the vertebrae 20 for attaching the implant 10 to the vertebrae 20 can avoid some or all of the shortcomings associated with the use of pedicle screws.

The implant 10 is shaped to fit into the intervertebral space 24 located between the spinous processes 36 of adjacent vertebrae 20. The implant 10 is configured to be expanded during surgery to allow distraction, or widening, of the intervertebral space 24 and/or the space occupied by the intervertebral disc 22 (the intervertebral disc 22 can be removed if desired). The distraction of the intervertebral space 24 and/or the space occupied by the intervertebral disc 22 can widen the intervertebral space 24 and the neural foramen 28 to restore them to healthy heights, which may have decreased in size during degeneration of a patient's spine. The distraction of the intervertebral space 24 and/or the space occupied by the intervertebral disc 22 can decompress the spinal canal or the nerve roots, which may have become compressed due to degeneration of the vertebrae 20.

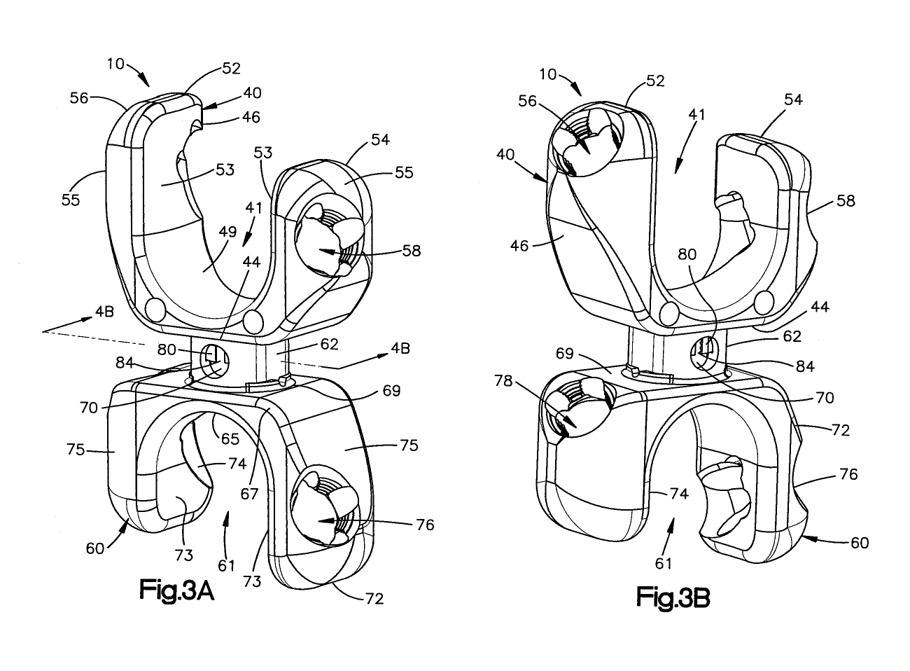

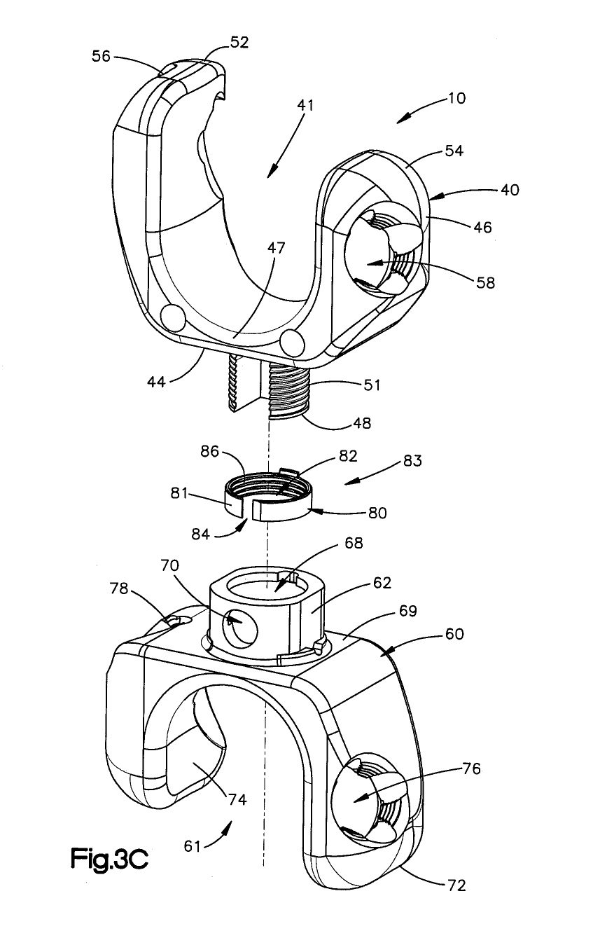

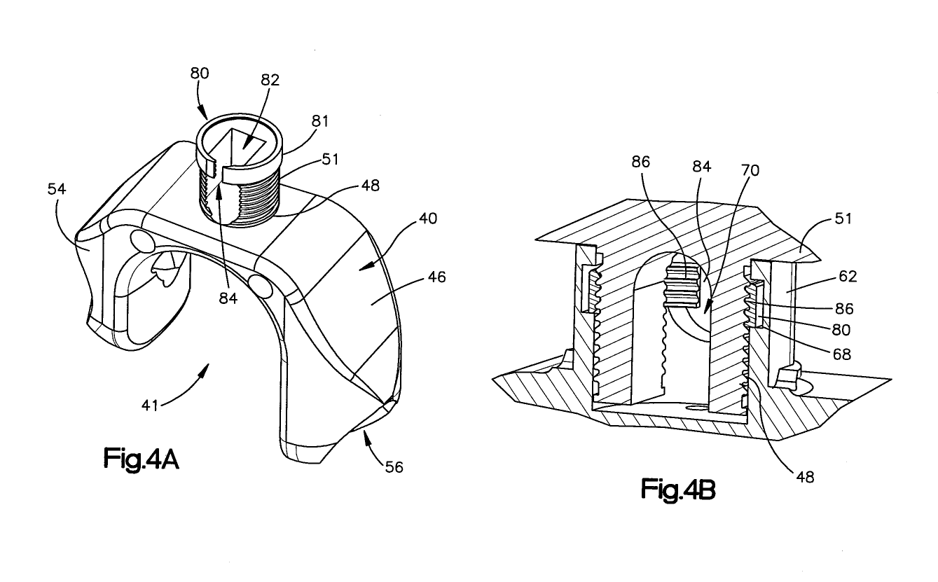

Referring now to FIGS. 3A-4B, the cranial fixator 40 and the caudal fixator 60 are longitudinally moveable relative to each other to allow the implant 10 to be longitudinally expandable in the cranial-caudal direction.

The cranial fixator 40 includes a fixator body 46 having a base 47, and first and second wings 52 and 54 extending longitudinally up from laterally opposing ends of the base 47. The wings 52 and 54 define respective inner surfaces 53 and outer surfaces 55. The first wing 52 includes a first bone screw aperture 56 extending through the first wing 52 and configured to receive a bone screw 16. The second wing 54 includes a second bone screw aperture 58 extending through the second wing 54 and configured to receive a bone screw 16. The base 47 defines a rounded top surface 49 and an opposing substantially planar bottom surface 44, though it should be appreciated that the surfaces 44 and 49 could assume any geometric configuration as desired. The inner surfaces 53 of the wings 42 and 54 along with the top surface 49 of the base 47 define, in combination, an upwardly oriented, generally u-shaped opening 41.

The fixator body 46 further includes a generally cylindrical core 51 extending longitudinally downward from the bottom surface 44 of the base 47. The core 51 includes an engagement member that can be configured as at least one ratchet ridge 48 such as a plurality of ratchet ridges 48 that extend outwardly from the outer surface 45 of the core 51 in the lateral-transverse plane of the implant 10.

The caudal fixator 60 includes a fixator body 66 having a base 67, and first and second wings 72 and 74 extending longitudinally down from laterally opposing ends of the base 67. The wings 72 and 74 define respective inner surfaces 73 and outer surfaces 75. The first wing 72 defines a first bone screw aperture 76 extending through the first wing 72 and configured to receive a bone screw 16. The second wing 74 defines a second bone screw aperture 78 extending through the second wing 74 and configured to receive a bone screw 16. The base 67 defines a rounded bottom surface 65 and an opposing substantially planar top surface 69, though it should be appreciated that the surfaces 65 and 69 could assume any geometric configuration as desired. The inner surfaces 73 of the wings 72 and 74 along with the bottom surface 65 of the base 67 define, in combination, a generally u-shaped opening 61.

The caudal fixator body 66 further includes a generally cylindrical socket 62 extending longitudinally upward from the top surface 69 of the base 67 of the fixator body 66. The socket 62 includes a generally cylindrical channel 68 that is configured to receive the circlip 80. The socket 62 defines an access aperture 70 extending therethrough that is configured to allow access to widen the circlip 80 as desired.

Referring to FIG. 3C in particular, the circlip 80 includes a generally annular body 81 that defines a generally cylindrical internal void 82. An access gap 84 extends through the body 81, and is positioned so as to be in alignment with the access aperture 70 of the socket 62 during use. The circlip 80 includes an engagement member that is complementary to the engagement member of the core 51 and configured to engage the core 51 so as to fix the longitudinal position of the cranial fixator 40 relative to the caudal fixator 60. For instance, the engagement member of the circlip 80 can be configured as at least one ratchet ridge 86 such as a plurality of ratchet ridges 86 that extend inwardly in the lateral-transverse plane of the implant 10. When the circlip 80 is disposed inside the channel 68, the annular body 81 compresses against the core 51, thereby causing the ratchet ridges 86 to mate with the ratchet ridges 48 of the cranial fixator 40. Engagement of the ratchet ridges 48 and 86 joins the cranial and caudal fixators 40 and 60 at a fixed height. As will be described in more detail below, disengagement of the ratchet ridges 48 and 86 allows the height of the implant to be adjusted. Thus, the core 51, the socket 62, and the circlip 80 define a locking mechanism 83 that selectively allows the fixators 40 and 60 to expand from an initial first height to a second desired height, and subsequently locking the fixators 40 and 60 at the second desired height.

An osseous integration promoter can be applied to the inner surface of the U-shaped opening 61. For instance, the U-shaped opening 61 can be coated or treated with macro-porous Titanium, or the surface can be enhanced with an anodic plasma-chemical process.

Referring again to FIGS. 3A-4B, the u-shaped opening 41 of the cranial fixator 40 and the u-shaped opening 61 of the caudal fixator 60 are configured to approximately correspond to the shape of spinous processes 36 in the lumbar spine. Accordingly, the openings 41 and 61 are configured to receive the respective spinous processes 36. In other embodiments, the u-shaped opening 41 of the cranial fixator 40 and the u-shaped opening 61 of the caudal fixator 60 can be configured to receive spinous processes in other regions of the vertebral column 12, including for example, the cervical spine.

The installed longitudinal height of the implant 10 will depend on the desired distance between the spinous processes 36 of adjacent vertebrae 20 in the spinal motion segment 14 to be treated. When the implant 10 is first inserted into a patient, the implant 10 can be in a fully collapsed position, in which the implant 10 has a minimum height, whereby the core 51 of the cranial fixator 40 is fully inserted into the socket 62 of the caudal fixator 60. Inserting the implant 10 into a patient in the fully collapsed position may allow the implant 10 to be inserted into a patient through a relatively small incision, thereby helping to minimize the degree of invasiveness of the spinal surgery, compared to inserting the implant 10 in an expanded position.

After the implant 10 is inserted into a patient, the implant 10 can be longitudinally expanded to the desired longitudinal height or the desired height of the intervertebral space 24 in the spinal motion segment 14 to be treated.

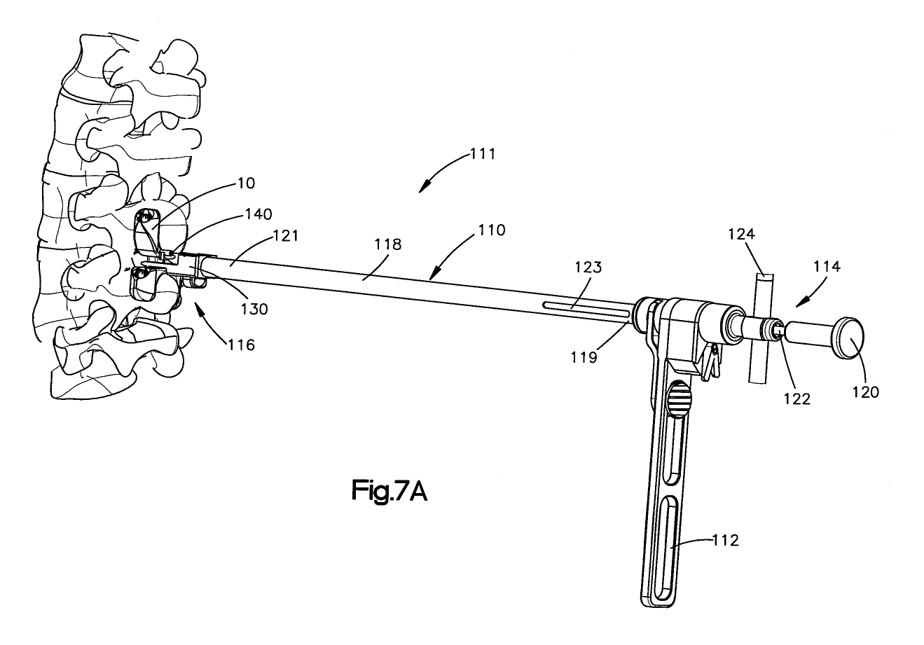

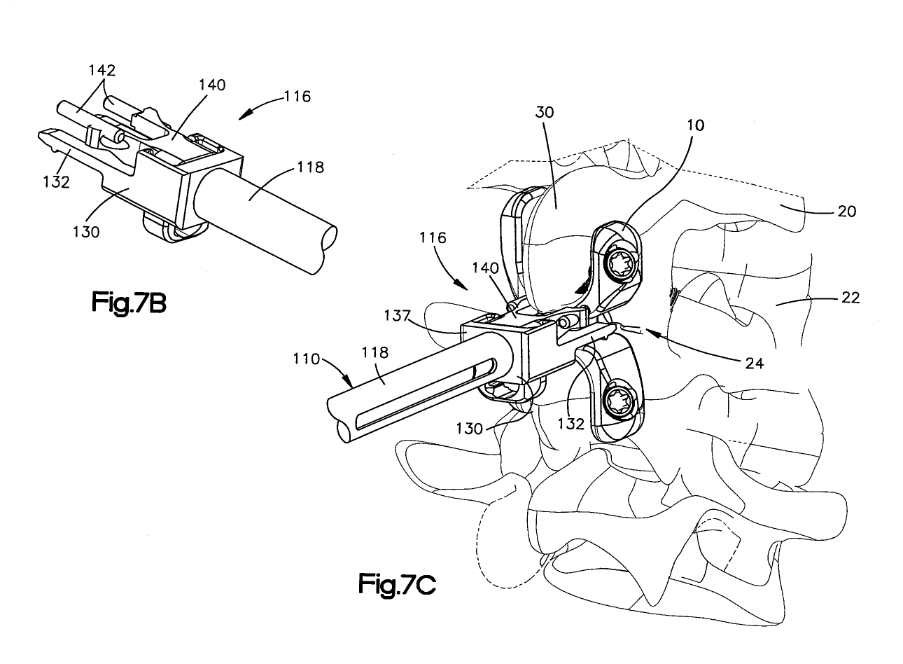

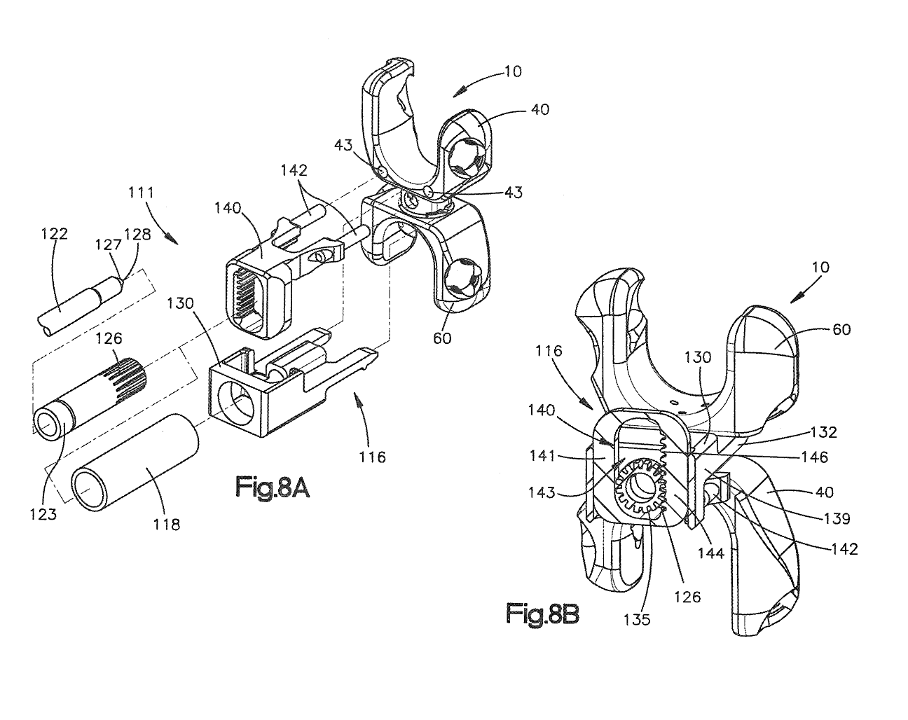

To expand the longitudinal height of the implant 10, the ratchet ridges 86 of the circlip 80 are disengaged from the ratchet ridges 48 of the cranial fixator 40. Accordingly, a tool (such as the tip of an insertion device 110 shown in FIGS. 7A-8B) is inserted into the access gap 84 through the access aperture 70 to widen or expand the internal void 82 of the circlip 80. When the circlip 80 is widened such that it expands inside of the channel 68, the ratchet ridges 86 release from engagement with the ratchet ridges 48 of the cranial fixator 40, thereby permitting the cranial fixator 40 to be moved longitudinally upward and downward relative to the caudal fixator 60. The upward movement of the cranial fixator 40 relative to the caudal fixator 60 causes the core 51 of the cranial fixator 40 to begin to withdraw from the socket 62 of the caudal fixator 60, such that the longitudinal height of the implant 10 is increased.

When the cranial fixator 40 has moved upward relative of the caudal fixator 60 such that the implant 10 has achieved the desired height, the circlip 80 can be released by removing the insertion device 110, thereby allowing the internal void 82 of the circlip 80 to return to its initial size, which causes the ratchet ridges 86 to again engage the ratchet ridges 48 of the cranial fixator 40. When the ratchet ridges 86 of the circlip 80 re-engage the ratchet ridges 48 of the cranial fixator 40, the height of the implant 10 is fixed at the desired height.

Although the cranial fixator 40 is shown in the Figures as being located above the caudal fixator 60 along the caudocranial axis C-C, in other embodiments, the implant 10 may be installed upside-down with respect to the illustrated orientation, such that the cranial fixator 40 is located below the caudal fixator 60 along the caudocranial axis C-C.

Although the cranial fixator 40 is illustrated as including a cylindrical core 51 and the caudal fixator 60 is shown as including a socket 62, in other embodiments, the cranial fixator 40 may include a socket, and the caudal fixator 60 may include a cylindrical core that is adapted to longitudinally slide into the socket of the cranial fixator 40.

Although the caudal fixator 60 is illustrated as including a single access aperture 70 extending therethrough in the transverse direction T, in other embodiments, the access aperture 70 may be circumferentially oriented in any direction in the lateral-transverse plane of the implant 10. The caudal fixator can further include a plurality of access apertures if desired. In such embodiments wherein the access aperture 70 has an alternate orientation, the access gap 84 of the circlip 80 can be circumferentially oriented to align with and be accessed through the access aperture 70.

If it is later desired to reduce the height of the implant 10, the circlip 80 can be widened again by inserting the insertion device 110 into the access gap 84 through the access aperture 70, to widen the internal void 82 of the circlip 80. When the circlip 80 is widened such that it expands inside of the channel 68, the ratchet ridges 86 release from engagement with the ratchet ridges 48 of the cranial fixator 40, thereby permitting the cranial fixator 40 to be moved longitudinally downward relative to the caudal fixator 60. When the cranial fixator 40 has moved downward such that the implant 10 has achieved the desired height, the circlip 80 can be released by removing the tool, thereby allowing the internal void 82 of the circlip 80 to return to its initial size, causing the ratchet ridges 86 to re-engage the ratchet ridges 48 of the cranial fixator 40.

It should be appreciated that the locking mechanism 83 has been illustrated in accordance with one embodiment, and that the locking mechanism can define alternative structure that is configured to allow the fixators 40 and 60 to expand from an initial height to a desired height, and subsequently lock the fixators 40 and 60 at the desired height.

The cranial fixator 40 and the caudal fixator 60 can be made from any material suitable for use as an implant inside of a patient. For example, the cranial fixator 40 and the caudal fixator 60 can be made from any metal can be used that is suitable for use as a long-term load-bearing implant, such as titanium. The cranial fixator and/or the caudal fixator 60 can be made from one or more elastic polymers that are biostable (not resorbable), including for example, PCU and/or similar elastomeric thermoplastic polymers. The cranial fixator and/or the caudal fixator 60 can be made from one or more radiolucent polymers, including for example, PEEK or carbon fiber reinforced PEEK.

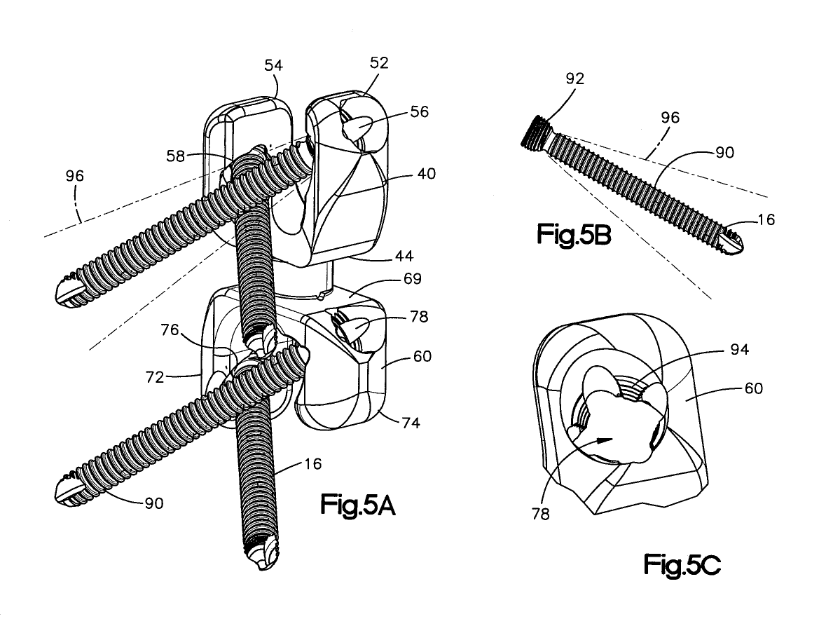

Referring now to FIGS. 5A-5C, the first wing 52 and the second wing 54 of the cranial fixator 40 and the first wing 72 and the second wing 74 of the caudal fixator 60 include asymmetrically-located respective first bone screw apertures 56 and 76 and second bone screw apertures 58 and 78. The first bone screw apertures 56 and 76 and the second bone screw apertures 58 and 78 are adapted to permit translaminar bone screws 16 to attach the implant 10 to the vertebrae 20 by passing through the laminae 30 of the vertebrae 20. The asymmetric relative positions of the first bone screw apertures 56 and 76 compared with the second bone screw apertures 58 and 78 prevents interference of the bone screws 16 as they are inserted into the laminae 30 of the respective vertebrae 20.

As illustrated, the first bone screw aperture 56 and 76 are located at a greater longitudinal distance from the respective bottom 44 of the cranial fixator 40 and the top 69 of the caudal fixator 60 than the second bone screw apertures 58 and 78. In other embodiments, the second bone screw aperture 58 and 78 can be located at a greater longitudinal distance from the respective bottom 44 of the cranial fixator 40 and the top 69 of the caudal fixator 60 than the first bone screw apertures 56 and 76.

In accordance with an alternative embodiment, the first bone screw aperture 56 and 76 and the second bone screw apertures 58 and 78 are located at approximately the same longitudinal distance from the respective bottom 44 of the cranial fixator 40 and the top 69 of the caudal fixator 60. In this embodiment, the range of insertion angles of the first bone screw aperture 56 and 76 can be sufficiently different than the range of insertion angles of the second bone screw aperture 58 and 78, such that interference of the bone screws 16 in the laminae 30 is avoided.

As can be seen in FIGS. 5B and 5C, each bone screw 16 and respective first bone screw apertures 56 and 76 and second bone screw apertures 58 and 78 includes a multi-axial locking screw mechanism. Each bone screw 16 includes a threaded shaft 90 and a threaded head 92. Each threaded head 92 has a substantially spherical shape. Each first bone screw aperture 56 and 76 and second bone screw aperture 58 and 78 includes tapped portions 94 that are configured to only partially bear the threaded head 92 of a bone screw 16.

The combination of the threaded spherical head 92 of each bone screw 16 and the tapped portions 94 that are configured to only partially bear the threaded head 92 result in the bone screws 16 being capable of variable insertion angles 96 relative to the respective first bone screw apertures 56 and 76 and second bone screw apertures 58 and 78. Additional disclosure related to multi-axial locking screw mechanisms are shown and described in co-pending U.S. provisional patent application No. 61/181,149 filed May 26, 2009, the disclosure of which is hereby incorporated by reference as if set forth in its entirety herein.

The multi-axial locking screw mechanism provided by the first bone screw apertures 56 and 76 and second bone screw apertures 58 and 78 allows a surgeon to insert the respective bone screws 16 at variable insertion angles 96. Such variable insertion angles 96 can allow the surgeon to direct the screw shafts in a direction as desired to avoid contact between bone screws 16 when they are inserted into the laminae 30 of the vertebrae 20, and to further avoid penetration of the bone screws 16 into the vertebral foramen 26 and the neural foramen 28 and contact with the spinal canal or the nerve roots.

The locking feature of the multi-axial locking screw mechanism included in each bone screw 16 and respective first bone screw apertures 56 and 76 and second bone screw apertures 58 and 78 allows the implant 10 to carry the loads applied to the spinal motion segments 14 of the vertebral column 12, thereby allowing the implant 10 to be a stable treatment for lumber posterior fusion.

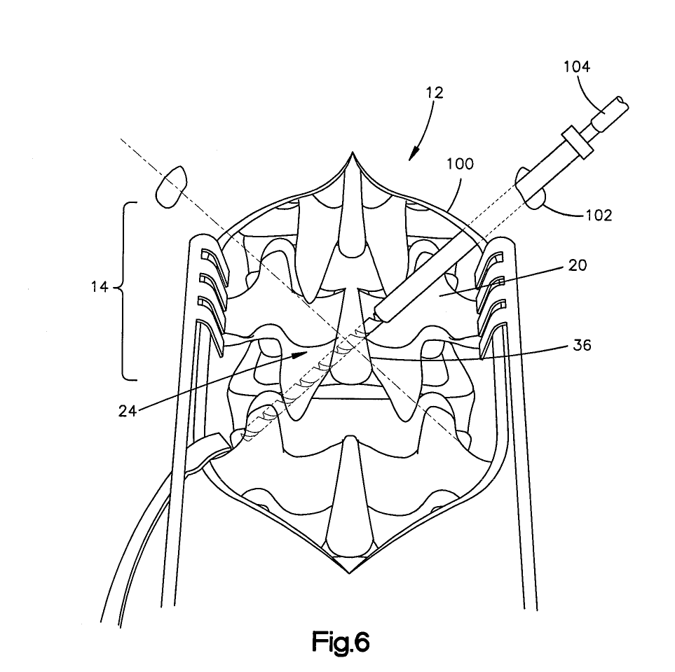

Referring now to FIG. 6, the implant 10 can be inserted into a patient through a relatively small median incision 100 along the lumber portion of the vertebral column 12, near the desired spinal motion segments 14 for installation of the implant 10. The bone screws 16 can be inserted into the patient through respective stab incisions 102, through which a drill 104 can provide pilot holes in the laminae 30 of the vertebrae 20 for insertion of the bone screws 16. The implant 10 can be installed into a patient using a translaminar screw fixation technique as known by one having ordinary skill in the art. In some embodiments, cannulated bone screws cam be used with guide wires to assist in the insertion of the implant 10 into the patient.

Installing the implant 10 into the intervertebral space 24, rather than installing an implant into the space occupied by an intervertebral disc 22, can allow a surgeon to install the implant 10 into a posterior incision (which is less invasive to the patient) rather than into an anterior incision (which is more invasive to the patient). Also, installing the implant 10 into the laminae 30 of the vertebrae 20 rather than into the pedicles 34 of the vertebrae 20 avoids major muscle delamination from the vertebrae 20 that is common when installing pedicle screws.

Referring now to FIGS. 7A-8B, the implant 10 can be inserted into a patient using an insertion device 110. The implant 10 and the insertion device 110 may together define an intervertebral implant system 111. The insertion device 110 includes a handle 112 configured to grip the insertion device 110, a control interface 114 configured to engage and release the circlip 80 and further configured to and set the height of the implant 10, and an expandable body 116 configured to hold and position the implant 10. A cannulated central tube 118 defines a proximal end 119 that is connected to the control interface 114, and an opposing distal end 121 that is connected to the expandable body 116.

The central tube 118 retains a translation rod 122 that is surrounded by an outer sleeve 123. The outer sleeve 123 is connected at its distal end to a cannulated pinion 126 that presents teeth 135. Alternatively, the outer sleeve 123 could be integrally coupled to the pinion 126. The translation rod 122 extends through the pinion 126 and defines an actuator, such as an engagement tip 128, that can define a pair of opposing beveled surfaces 127 that flare outward along a direction from the distal end 121 of the central tube toward the proximal end 119 of the central tube 118.

The control interface 114 includes a translation plunger 120 coupled to the rod 122. Translation of the plunger 120 along the transverse direction T causes the rod 122 to likewise translate along the transverse direction T. Forward translational motion of the rod 122 inserts the tip 128 through the access aperture 70 in the socket 62 and into the access gap 84 of the circlip 80. The beveled outer surfaces 127 cause the circlip 80 to expand, thereby disengaging the ratchet ridges 86 of the circlip 80 from the ratchet ridges 48 of the cranial fixator 40. Rearward movement of the plunger 120 removes the tip 128 from the access gap 84, which thereby allows the circlip 80 to collapse to its initial configuration whereby the ratchet ridges 86 and 48 engage. In this regard, the tip 128 can be referred to as an actuator that can move from a first position that causes the circlip 80 to disengage the ratchet ridges 86 from the ratchet ridges 48, thereby allowing at least one of the cranial and caudal fixators 40 and 60 to move relative to the other along the longitudinal axis, to a second position that prevents the cranial and caudal fixators 40 and 60 from moving longitudinally relative to each other.

With continuing reference to FIGS. 7A-8B, the expandable body 116 includes a cranial slider housing 140 and a caudal support housing 130 that receives the cranial slider housing 140. The support housing 130 defines a housing body 137 that is coupled to the distal end 121 of the central tube 118. The support housing 130 includes a pair of laterally spaced vertical arms 139, and a pair of spaced caudal fingers 132 that extend forward from the housing body vertical arms 139. The caudal fingers 132 are configured to secure the caudal fixator 60 around the outside of the cylindrical socket 62.

The slider housing 140 includes a body 141 and a pair of cranial fingers 142 that extend forward from the body 141 and are configured to retain the cranial fixator 40 therebetween. In particular, the cranial fingers 142 secure the cranial fixator 40 by extending into transverse apertures 43 extending into the cranial fixator 40. The body 141 defines an internal opening 143 that receives the pinion 126. The body 141 includes a rack 144 that presents teeth 146 projecting into the opening that mate with the teeth 135 of the pinion 126. The control interface 114 includes a rotation actuator 124 configured to impart rotational motion onto the cannulated pinion 126, which causes the teeth 135 of the pinion 126 to drive the rack 144, and thus the slider housing 140, to translate in the caudal-cranial direction within the support housing 130, thereby expanding the tip 116.

During operation, a surgeon can install the implant 10 into a patient in a fully collapsed position, in which the implant 10 has a minimum height, whereby the core 51 of the cranial fixator 40 is fully inserted into the socket 62 of the caudal fixator 60, so that the size of the median incision can be minimized. To install the implant 10 into a patient, the surgeon inserts the cranial fixator 40 between the cranial fingers 142, and the caudal fixator 60 between the caudal fingers 132, such that the fingers 132 and 142 retain the implant 10 in the manner described above. The surgeon then grips the handle 112 and moves the implant 10 into the median incision 100 with the insertion device 110. Once the implant 10 is positioned into the intervertebral space 24 in a desired spinal motion segment 14, the surgeon attaches the caudal fixator 60 to the lamina 30 of the lower vertebra 20, using bone screws 16 to lock the caudal fixator 60 to the lamina 30.

Once the caudal fixator 60 is attached to the lamina 30, the surgeon can begin to increase the vertical height of the implant 10 by longitudinally moving the cranial fixator 40 relative to the caudal fixator 60. The surgeon first releases the circlip 80 from the cranial fixator 40 by moving the translation plunger 120 along the transverse direction T toward the implant 10. As the translation plunger 120 moves along the transverse direction T, the tip 128 of the rod 122 is inserted through the access aperture 70 in the socket 62 into the access gap 84 of the circlip 80, thereby causing the beveled surfaces 127 to disengage the ratchet ridges 86 of the circlip 80 from the ratchet ridges 48 of the cranial fixator 40.

Once the circlip 80 is disengaged from the cranial fixator 40, the surgeon can raise the cranial fixator 40 relative to the caudal fixator 60 by rotating the rotation actuator 124 clockwise. When the rotation actuator 124 is rotated clockwise, the cannulated pinion 126 is rotated clockwise against the rack 144, thereby moving the slider housing 140 upward along the longitudinal direction L relative to the support housing 130 and expanding the tip 116. As the cranial slider housing 140 of the expandable body 116 moves upward along the longitudinal direction L relative to the caudal support housing 130, the cranial fixator 40 moves upward along the longitudinal direction L relative to the caudal fixator 60.

Once the implant 10 has reached the desired height, whereby the cranial fixator 40 has moved to the desired longitudinal position relative to the caudal fixator 60, the surgeon attaches the cranial fixator 40 to the lamina 30 of the upper vertebra 20, using bone screws 16 to lock the cranial fixator 40 to the lamina 30. Once the implant 10 is completely secured to the laminae 30 of the vertebrae 20, the surgeon pulls the insertion device 110 out of engagement with the implant 10 and removes the insertion device 110 from the median incision 100, thereby completing installation of the implant 10 in the patient. The position of the implant 10 in the intervertebral space 24 in the desired spinal motion segment 14 can be evaluated with diagnostic tests, such as x-rays.

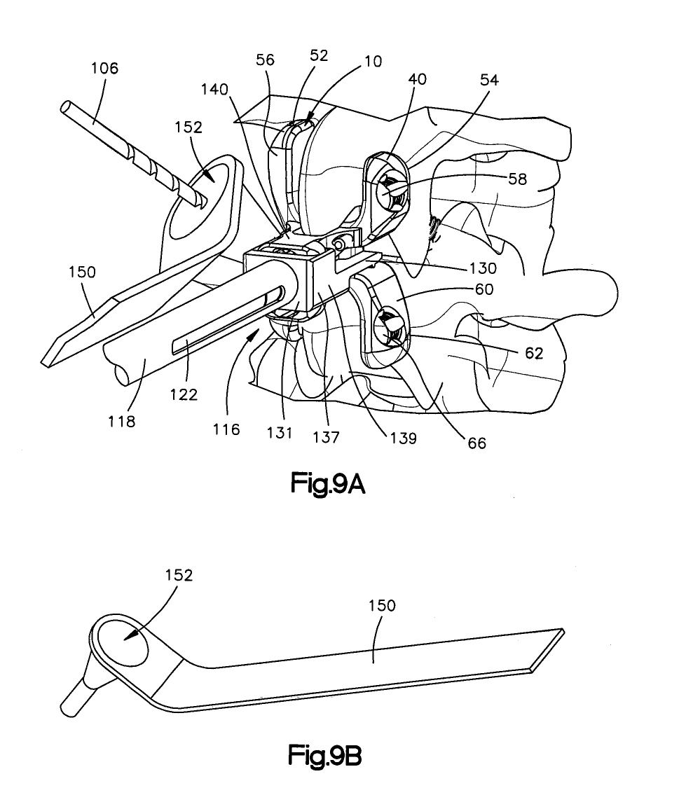

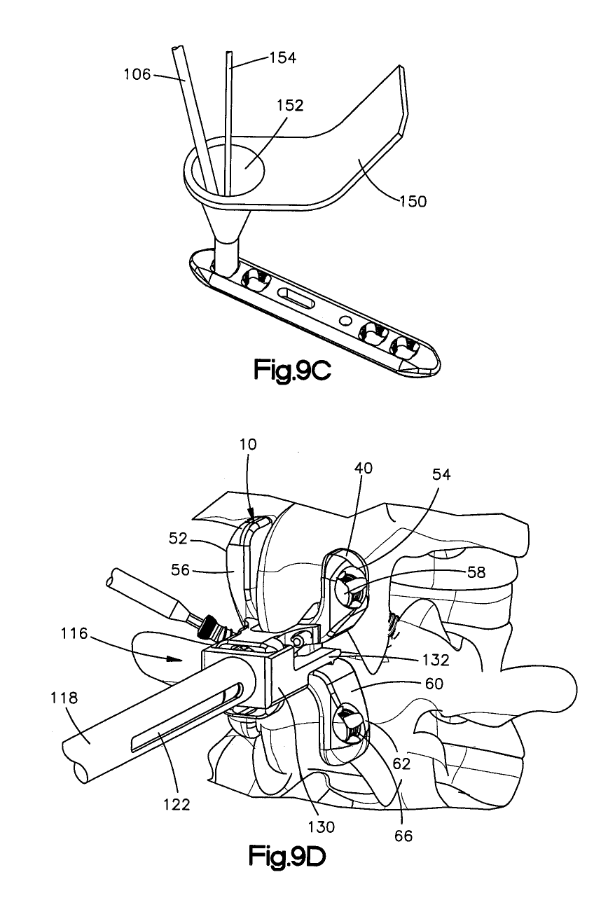

Referring now to FIGS. 9A-9D, before attaching the implant 10 to the laminae 30 of the vertebrae 20, a surgeon can use a drill 104 to provide pilot holes in the laminae 30 for insertion of the bone screws 16. To drill the pilot holes in the laminae 30, an aiming device 150 can be inserted into the patient through the median incision 100, where the surgeon is able to view the intervertebral space 24 in the desired spinal motion segment 14 where the implant 10 will be installed. A drill bit 106 of the drill 104 is inserted through the stab incisions 102 into an aperture 152 of the aiming device 150.

The aperture 152 of the aiming device 150 limits the angle of insertion of the drill bit 106, while providing variable insertion angles 154 of the multi-axial aiming device 150. The variable insertion angles 154 of the aperture 152 of the aiming device 150 can be configured to approximately match the variable insertion angles 96 of the multi-axial locking screw mechanism included in each bone screw 16 and respective first bone screw apertures 56 and 76 and second bone screw apertures 58 and 78. If the variable insertion angles 154 of the multi-axial aiming device 150 are approximately matched to the variable insertion angles 96 of the multi-axial locking screw mechanism, then it will be likely that the drilled pilot holes in the laminae 30 will be able to accommodate the desired insertion angle of the bone screws 16. Once the pilot holes are drilled in the laminae 30, a screwdriver 156 can be inserted through the stab incisions 102 to insert the bone screws 16 into the laminae 30.

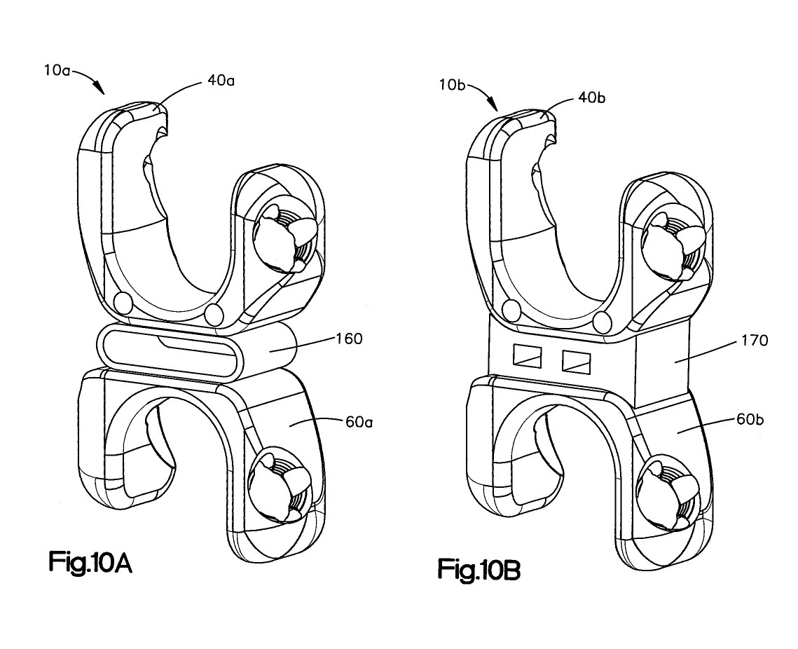

Referring now to FIG. 10A, a second embodiment expandable intervertebral implant 10a for posterior lumbar intervertebral stabilization includes a cranial fixator 40a, a caudal fixator 60a that is moveable relative to the cranial fixator 40a, and a blade spring 160 located between cranial fixator 40a and caudal fixator 60a that is biased to an open position such that it resists compressive forces that move cranial fixator 40a and caudal fixator 60a toward each other. Although a blade spring 160 is shown in FIG. 10A, any type of spring or compressible device can be used to resist compressive forces between the cranial fixator 40a and the caudal fixator 60b.

The implant 10a is suitable for installation into the intervertebral space 24 of the spinal motion segment 14 of the vertebral column 12 shown in FIGS. 1-2C by attaching the implant 10a to the laminae 30 of adjacent vertebrae 20 by bone screws 16. Such an embodiment can be used, for example, when a surgeon intends to dampen the motion of a desired spinal motion segment 14 and restore the height of the desired spinal motion segment 14.

The implant 10a can be inserted in a first position, having a first height, into a patient through the median incision 100 shown in FIG. 6, and the implant 10a can expand to a second, or expanded, position having a second height that is greater than the first height when the surgeon releases compressive pressure from cranial fixator 40a and caudal fixator 60a, such that the cranial fixator 40a and the caudal fixator 60a can be attached to adjacent spinous processes 36 by bone screws 16 as shown in FIGS. 9A-9D.

Referring now to FIG. 10B, a third embodiment expandable intervertebral implant 10b for posterior lumbar intervertebral stabilization includes a cranial fixator 40b, a caudal fixator 60b that is moveable relative to the cranial fixator 40b, and an elastic dampener 170 located between cranial fixator 40b and caudal fixator 60b that is biased to an open position such that it resists compressive forces that move cranial fixator 40b and caudal fixator 60b toward each other.

As shown in FIG. 10B, elastic dampener 170 is an elastomer or polymer that can dampen the motion of the spinal motion segment 14 with viscoelastic progression. In other embodiments, any type of elastic dampener or compressible device can be used to resist compressive forces between the cranial fixator 40b and the caudal fixator 60b.

The implant 10b is suitable for installation into the intervertebral space 24 of the spinal motion segment 14 of the vertebral column 12 shown in FIGS. 1-2C by attaching the implant 10b to the laminae 30 of adjacent vertebrae 20 by bone screws 16. Such an embodiment can be used, for example, when a surgeon intends to dampen the motion of a desired spinal motion segment 14 and restore the height of the desired spinal motion segment 14.

The implant 10b can be inserted in a compressed position into a patient through the median incision 100 shown in FIG. 6, and the height of the implant 10b can expand when the surgeon releases compressive pressure from cranial fixator 40b and caudal fixator 60b, such that the cranial fixator 40b and the caudal fixator 60b can be attached to adjacent spinous processes 36 by bone screws 16 as shown in FIGS. 9A-9D.

When compressive pressure is released from implant 10b, the restoration of the height of the spinal motion segment 14 is achieved slowly after the compressive pressure is released. For example, this slower restoration of the height of the spinal motion segment 14 can be advantageous for an elderly patient with brittle or sclerotic bone quality.

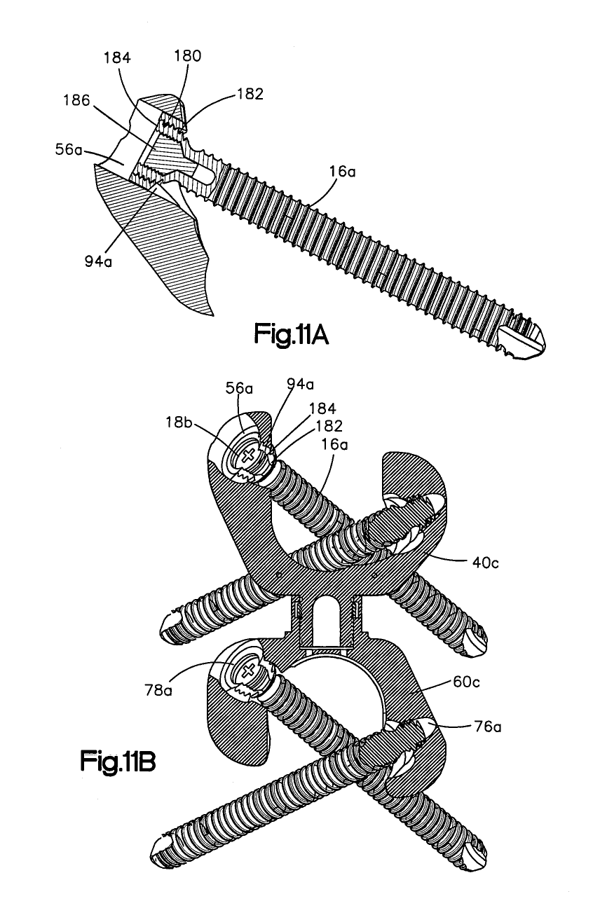

Referring now to FIGS. 11A and 11B, a bone screw 16a includes a multi-axial fixation mechanism comprising an expanding ring 180 located around a ball-shaped head 182 defining deflectable head portions 184, and an expansion screw 186 located within the head 182. Each bone screw 16a is configured to lock into respective first bone screw apertures 56a and 76a and second bone screw apertures 58a and 78a that include untapped internal surfaces 94a that are configured to mate with the expanding ring 180.

The bone screw 16a and the bone screw apertures 56a, 58a, 76a, and 78a that include untapped internal surfaces 94a (shown in FIGS. 11A and 11B) are suitable for use as an alternative to the bone screw 16 and bone screw apertures 56, 58, 76, and 78 that include tapped portions 94 (shown in FIGS. 5A-5C) in installing any of the intervertebral implants 10, 10a, or 10b into the intervertebral space 24 of the spinal motion segment 14 of the vertebral column 12 shown in FIGS. 1-2C by attaching the implant to the laminae 30 of adjacent vertebrae 20 by bone screws 16a.

To use bone screws 16a to install an implant 10, 10a, or 10b into the laminae 30 of adjacent vertebrae 20, a surgeon first drills one or more a pilot holes in into the laminae 30 with a drill bit, as shown in FIG. 9A. Once the pilot holes are drilled, the surgeon orients each bone screw 16a to a desired angle relative to the implant 10, 10a, or 10b. Similar to the bone screw 16, the bone screw 16a is configured to provide a surgeon with variable insertion angles 96 relative to the respective bone screw apertures as shown in FIG. 5A.

Once the desired angle for each bone screw 16a is chosen, the surgeon advances each bone screw 16a through the respective bone screw aperture and into the laminae 30. To lock each bone screw 16a into either the cranial fixator 40c or the caudal fixator 60c, the surgeon advances the respective expansion screw 186, which deflects the deflectable head portions 184, thereby widening the respective head 182 and locking the head 182 against the expanding ring 180, which becomes locked against the untapped internal surfaces 94a.

The foregoing description is provided for the purpose of explanation and is not to be construed as limiting the invention. While the invention has been described with reference to preferred embodiments or preferred methods, it is understood that the words which have been used herein are words of description and illustration, rather than words of limitation. Furthermore, although the invention has been described herein with reference to particular structure, methods, and embodiments, the invention is not intended to be limited to the particulars disclosed herein, as the invention extends to all structures, methods and uses that are within the scope of the appended claims. Further, several advantages have been described that flow from the structure and methods; the present invention is not limited to structure and methods that encompass any or all of these advantages. Those skilled in spinal implant technology, having the benefit of the teachings of this specification, may effect numerous modifications to the invention as described herein, and changes can be made without departing from the scope and spirit of the invention as defined by the appended claims. Furthermore, any features of one described embodiment can be applicable to the other embodiments described herein. For example, any features or advantages related to the design of the cranial fixator or caudal fixator with respect to discussion of a particular expandable intervertebral implant embodiment can be applicable to any of the other expandable intervertebral implant embodiments described herein.

What is claimed:

1. An expandable intervertebral implant configured to be inserted into an intervertebral space defined between first and second vertebrae, the implant comprising:
a first fixator including a first fixator base, the first fixator configured to be attached to a lamina of the first vertebra;
a second fixator including a second fixator base, the second fixator configured to be attached to a lamina of the second vertebra;
a socket extending out from the second fixator base; and
a core extending out from the first fixator base and sized to be received in the socket, the core including an engagement member configured to releasably fix a position of the first fixator relative to the second fixator.

2. The expandable intervertebral implant as recited in claim 1, wherein the engagement member of the core comprises at least one ratchet ridge.

3. The expandable intervertebral implant as recited in claim 1, wherein the engagement member of the core comprises a plurality of ratchet ridges adjacent each other along a longitudinal direction.

4. The expandable intervertebral implant as recited in claim 1, wherein the first and second fixators are spaced from each other along a longitudinal direction when the implant is disposed in the intervertebral space, and the engagement member is configured to releasably fix the first fixator with respect to longitudinal movement relative to the second fixator.

5. The expandable intervertebral implant as recited in claim 4, whereby the implant is configured to be expanded after installation into the intervertebral space, such that the implant extends between spinous processes of the vertebrae.

6. The expandable intervertebral implant as recited in claim 5, wherein the first fixator further includes first and second fixator wings that extend longitudinally from the first fixator base, the first and second fixator wings defining an opening that is configured to receive a spinous process of the first vertebrae.

7. The expandable intervertebral implant as recited in claim 6, wherein the second fixator further includes first and second fixator wings that extend longitudinally form the second fixator base, the first and second fixator wings defining an opening that is configured to receive a spinous process of the second vertebrae.

8. The expandable intervertebral implant as recited in claim 7, wherein the first and second wings, of the first and second fixators each define a bone screw aperture configured to receive a bone screw.

9. The expandable intervertebral implant as recited in claim 8, wherein each bone screw aperture includes a tapped portion.

10. The expandable intervertebral implant as recited in claim 6, wherein the first and second wings each include a bone screw aperture that is asymmetrically-located with respect to the other.

11. The expandable intervertebral implant as recited in claim 6, wherein the first and second wings each include a bone a screw aperture, each bone screw aperture defining a range of insertion angles for a bone screw.

12. The expandable intervertebral implant as recited in claim 1, further comprising a circlip configured to fix the longitudinal position of the first fixator relative to the second fixator, the circlip including an engagement member, and configured to fit inside the socket, the circlip engagement member configured to mate with the engagement member of the core.

13. The expandable intervertebral implant as recited in claim 12, wherein the first and second fixators are spaced from each other along a longitudinal direction when the implant is disposed in the intervertebral space, and the engagement members of the core and the circlip are configured to releasably fix the first fixator with respect to the second fixator.

14. The expandable intervertebral implant as recited in claim 13, wherein the socket defines an access aperture that is configured to allow access to the circlip so that the circlip can be widened.

15. The expandable intervertebral implant as recited in claim 14, wherein the circlip includes a body and an access gap that extends through the body, the access gap configured to be in alignment with the access aperture of the socket when the circlip is positioned within the socket.

16. The expandable intervertebral implant as recited in claim 15, wherein both the access aperture and the access gap extend in a direction transverse to the longitudinal direction.

17. The expandable intervertebral implant as recited in claim 12, wherein the socket defines a cylindrical channel, and the circlip is received within the channel.

18. The expandable intervertebral implant as recited in claim 1, wherein the core is substantially cylindrical.

19. The expandable intervertebral implant as recited in claim 1, wherein the first fixator defines transverse apertures that are configured to receive fingers of an insertion device.

20. An expandable intervertebral implant system comprising:
an intervertebral implant configured to be inserted into an intervertebral space defined between adjacent vertebrae and attached to a spinous process of the adjacent vertebrae, the implant including a first fixator, a second fixator, and a locking mechanism that selectively allows the first and second fixators to expand from a first height to a second height; and
an insertion device configured to be coupled to the implant, the insertion device including an actuator that is configured to selectively engage the locking mechanism so as to selectively unlock the locking mechanism and allow the first and second fixators to expand from the first height to the second height.

21. The system as recited in claim 20, wherein the insertion device further includes an expandable body that is configured to expand the implant when the actuator is engaged with the locking mechanism.

22. The system as recited claim 20, wherein (i) the second fixator includes a second fixator base and a socket extending longitudinally from the second fixator base, and (ii) the first fixator includes a first fixator base and a core extending longitudinally from the first fixator base, the core including outwardly-extending ratchet ridges and configured to fit into the socket.

23. The system as recited in claim 22, wherein the implant further includes a circlip, the circlip including inwardly-extending circlip ratchet ridges and configured to fit inside the socket, the circlip ratchet ridges configured to mate with the ratchet ridges to thereby at least partially define the locking mechanism.

24. The system as recited in claim 23, wherein the socket defines an access aperture that is configured to allow access to the circlip so that the circlip can be engaged and widened by the actuator such that the circlip ratchet ridges disengage from the ratchet ridges of the core.

25. The system as recited in claim 24, wherein the circlip includes a body and an access gap that extends through the body, the access gap configured to be in alignment with the access aperture of the socket when the circlip is positioned within the socket.

26. The system as recited in claim 21, wherein (i) the first fixator defines a pair of transverse apertures, and the expandable body includes a slider housing having a pair of fingers that are configured to engage the transverse apertures of the first fixator, and (ii) the slider housing is configured to translate to thereby expand the implant when the fingers are engaged with the transverse apertures.

27. The system as recited in claim 26, wherein (i) the slider housing includes a slider housing body that defines an internal opening, and a rack defining teeth that project into the opening, (ii) the insertion device further includes a pinion defining teeth, the pinion extending into the opening such that the teeth of the pinion mate with the teeth of the rack, and (iii) rotation of the pinion causes the slider housing to translate in the longitudinal direction to thereby expand the implant.

28. The system as recited in claim 27, wherein the expandable body includes a support housing having a pair of fingers, the support housing fingers configured to secure the second fixator around the outside of the socket when the implant is coupled to the insertion device.

29. The system as recited in claim 20, wherein the actuator is an engagement tip.

30. A method of expanding an intervertebral implant for posterior lumbar intervertebral fusion of a spinal motion segment, the method comprising the steps of:
inserting the implant into an insertion device, the implant defining a longitudinal height, the implant including a first fixator having a core, and a second fixator having a socket that is configured to receive the core;
inserting the implant into an intervertebral space between vertebrae of the spinal motion segment, the vertebrae including a first vertebra and a second vertebra;
attaching the second fixator to a lamina of the second vertebra;
translating at least one of the first fixator and the second fixator relative to the other, thereby increasing the longitudinal height of the intervertebral implant;
fixing the position of the second fixator relative to the first fixator; and
attaching the first fixator to a lamina of the first vertebra.

31. The method as recited in claim 30, wherein the implant further defines a circlip configured to fix the longitudinal position of the second fixator relative to the first fixator, further comprising the steps of:
widening the circlip such that inwardly-extending ratchet ridges of the circlip are disengaged from outwardly-extending ratchet ridges of the first fixator core; and
releasing the circlip to engage the ratchet ridges of the circlip into the ratchet ridges of the first fixator core to thereby fix the position of the second fixator relative to the first fixator.
